(19) United States
(12) Patent Application Publication
(10) Pub. No.: US 2014/0305441 A1
(43) Pub. Date: Oct. 16, 2014

(54) Cushion for Crossed-Ankle and Other Body Part Support

(71) Applicant: Gregory Alan Porter, Lawrence, MA (US)

(72) Inventor: Gregory Alan Porter, Lawrence, MA (US)

(21) Appl. No.: 14/252,563

(22) Filed: Apr. 14, 2014

Related U.S. Application Data
(60) Provisional application No. 61/811,935, filed on Apr. 15, 2013.

(51) Int. Cl. A61F 5/01

(52) U.S. Cl. CPC A61F 5/0102
USPC 128/845

(57) ABSTRACT

A support and cushioning structure is disclosed. The support and cushioning structure is capable of supporting and cushioning the ankles in crossed-ankle positions. It includes a lower channel sized and positioned to allow a lower leg to pass through, and a top face with an elongate concave support for the calf of the upper leg and a symmetrical pair of concave ankle rests for the ankle of the upper leg. The support and cushioning structure may be comprised of a block of foam, such as urethane foam, and may be covered by a flexible cover.

### CROSS-REFERENCE TO RELATED APPLICATIONS

This application claims priority to U.S. Provisional Application No. 61/811,935, filed Apr. 15, 2013, the contents of which are incorporated by reference in their entirety.

### BACKGROUND OF THE INVENTION

1. Field of the Invention

In general, the invention relates to supports and cushions for human body parts, and more particularly to a support and cushion for crossed ankles and other body parts.

2. Description of Related Art

People often rest using their own bodies to support themselves. It is very common, for example, for a person to rest with legs extended and ankles crossed, or to support one's head with an arm. These are intuitively comfortable positions for most people. Unfortunately, remaining in these sorts of positions for more than a few minutes can fatigue muscles and cause nerve compression, resulting in uncomfortable “pins and needles” sensations and temporary loss in sensation in the limb.

A variety of limb supports and cushions exist, most of them for medical purposes. For example, U.S. Pat. No. 3,145,397 to Liftman discloses a leg support that can hold one leg elevated over the other. The support is particularly intended for medical uses, to relieve pressure during long convalescence. Other leg supports are designed simply to elevate one leg, for example. U.S. Pat. No. D424,698.

Relatively few solutions exist for those interested in supporting or cushioning their legs in the crossed-leg or crossed-ankle positions that many people find comfortable.

### SUMMARY OF THE INVENTION

Aspects of the invention relate to supports and cushions that are particularly useful for supporting the legs in crossed-ankle positions. In one embodiment, a support and cushion may have an open arch or channel that allows the support and cushion to rest on a lower leg. On the top face of the support and cushion, above the channel, concave supports are defined to support and cradle the calf and ankle of the upper leg, which can be crossed over the lower leg with the support and cushion between the two legs. The support and cushion may be made, for example, of a block of a foam, such as urethane foam.

Other aspects, features, and advantages of the invention will be set forth in the description that follows.

### BRIEF DESCRIPTION OF THE DRAWING FIGURES

The invention will be described with respect to the following drawing figures, in which like numerals represent like elements throughout the views, and in which:

FIG. 1 is a perspective view of a support and cushion according to one embodiment of the invention, shown in use with crossed ankles;

FIG. 2 is an elevational view of one side of the support and cushion of FIG. 1;

FIG. 3 is an elevational view of the other side of the support and cushion of FIG. 1

FIG. 4 is a perspective view of the support and cushion of FIG. 1, used to support the head overtop one arm;

FIG. 5 is an elevational view of a support and cushion according to another embodiment of the invention, illustrating an opening for the placement of hot and cold packs; and

FIG. 6 is a perspective view of yet another embodiment of a support and cushion according to the invention, illustrating a fabric cover.

### DETAILED DESCRIPTION

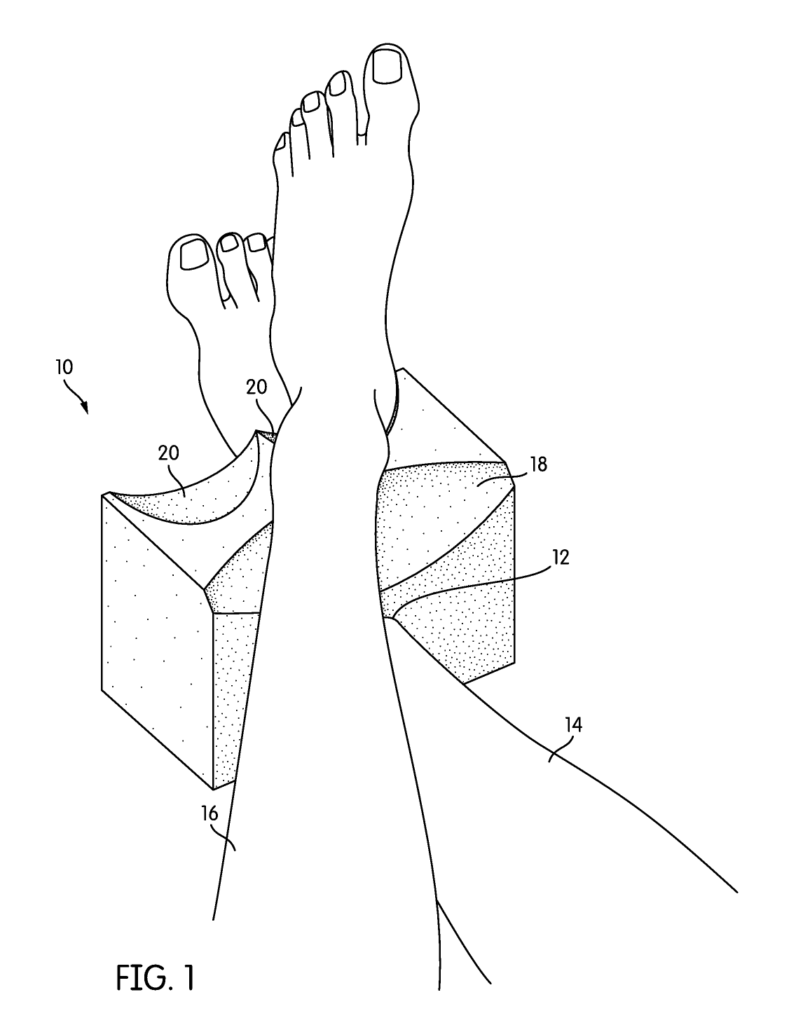

FIG. 1 is a perspective view of a support and cushion, generally indicated at 10, shown in use supporting a pair of legs in a crossed-ankle position. In the illustrated embodiment, the support and cushion 10 has the general shape of a rectangular block. As will be described below in more detail, the support and cushion 10 has a central opening or channel 12 that allows the lower of the two legs 14 to pass through it, such that the support and cushion 10 rests on the lower leg 14, while the upper leg 16 rests atop the support and cushion 10, typically in concave depressions or features provided specifically to support the leg and ankle of the upper leg 16.

More particularly, the support and cushion 10 has a concave depression 18 that spans the width of one of its top edges, and a symmetrical pair of approximately ankle-width concave depressions 20 along the opposite top edge of the support and cushion 10. The ankle-width depressions 20 and the As shown, the upper leg 16 crosses the lower leg 14 atop the cushion and is positioned such that its ankle rests in one of the depressions 20. The ankle-width depressions 20 allow the support and cushion 10 to be used regardless of whether the left leg or the right leg is the upper leg. Opposite one another as they are on the top face, the concave depression 18 and the two ankle-width concave depressions 20 are aligned in the same direction as the channel 12. Thus, the support and cushion 10 provides a more comfortable means of resting in crossed-ankle positions and potentially alleviates nerve compression and the uncomfortable sensations associated with it.

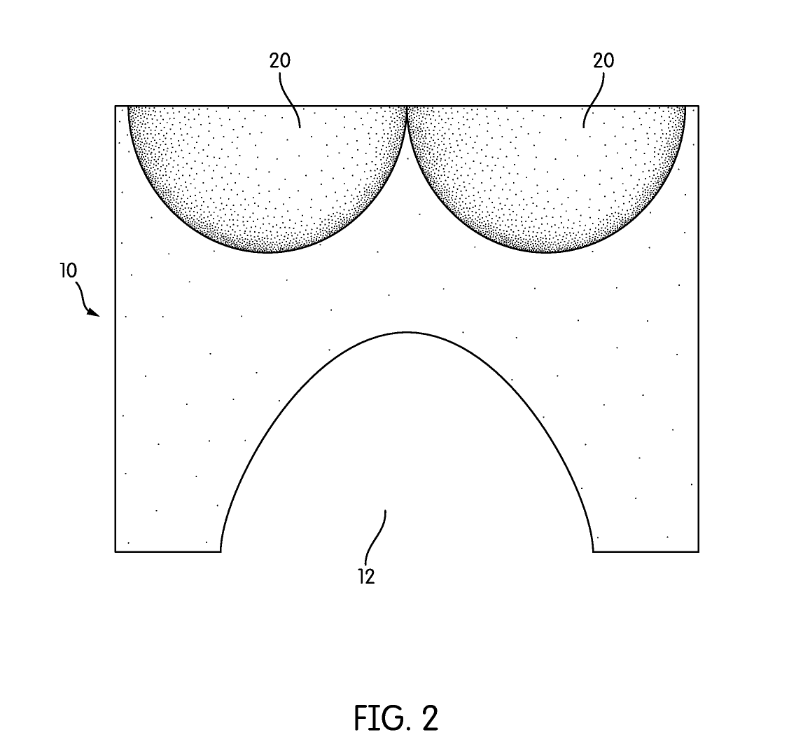

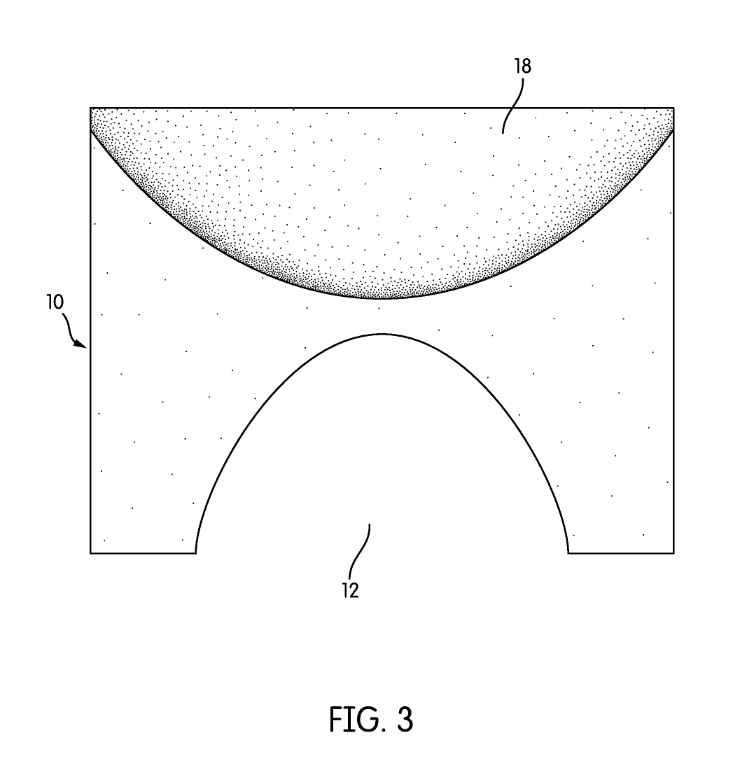

FIG. 2 is a side elevational view of the support and cushion 10 from one side, and FIG. 3 is a side elevational view of the support and cushion from the other side. As shown in FIGS. 2 and 3, the channel 12 of the illustrated embodiment is arch-shaped and of sufficient size to allow it to be placed over one leg proximate to the ankle However, in other embodiments, the channel 12 may be of a different shape, so long as it can accommodate a leg. The depressions 18, 20 are similarly of sufficient depth and size to allow the feet and ankles to rest comfortably in them. While the particular dimensions are not critical to the invention and may vary with leg size, foam density, and other factors, in one embodiment, the support and cushion 10 may be, for example, about 12 inches wide with a height and depth of about 7 inches. In that embodiment, the channel 12 may be about 3 inches high and about 5 inches wide, the concave depression may be about 9 inches wide and about 4 inches deep at its deepest point, and the two ankle-width depressions may be about 5 inches wide and about 3 inches deep at the deepest. Of course, the support and cushion 10 may be made in multiple sizes to accommodate different size legs and feet. However, one size of support and cushion 10 may support many sizes of legs and feet.

The overall shape of the cushion and support 10 is also not critical to the invention, and in other embodiments, it may have other shapes and other configurations. In particular, while the features 12, 18, 20 of the cushion and support 10 of the present invention are essentially subtractive—carved out of a larger block of material—that need not be the case in all embodiments. In other embodiments, the features may be additive, or positively defined without reference to a larger shape or block of material.

The support and cushion 10 may be made of many different types of conventional materials, including open- and closed-cell foams. As one example, the cushion and support may be made of a block of urethane foam of sufficient density to support and cushion the legs 14, 16. The foam may be a unitary block of material, or it may be assembled from sheets of material that are adhesively bonded or fused together. Although foam alone is likely to be sufficient in most embodiments and applications, in some cases, the support and cushion 10 could be reinforced in select locations with metal or plastic reinforcements.

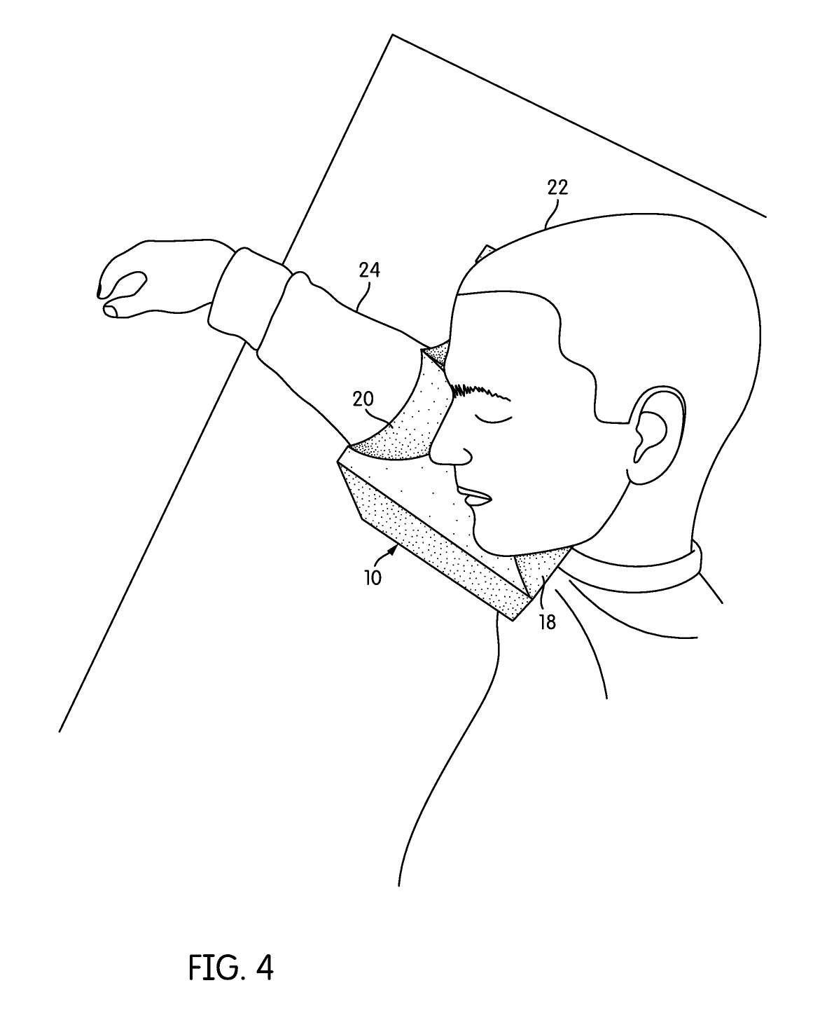

While the above disclosure focuses on the use of the support and cushion 10 to support crossed ankles and legs, it may be used in other ways as well. For example, FIG. 4 is a perspective view of the support and cushion 10 in use supporting a head 22 on its top surface with an arm 24 passing through the channel 12 under the head 22.

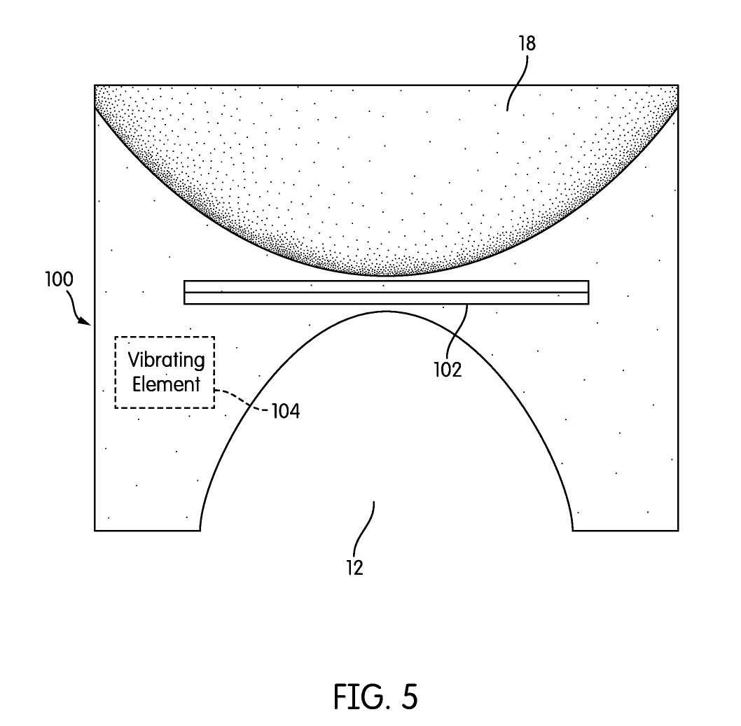

FIG. 5 is an elevational view of a support and cushion 100 according to another embodiment of the invention. The cushion 100 has the same basic features as the cushion 10 of FIGS. 1-4. Additionally, the cushion 100 includes an opening and pocket 102 below the top surface of the cushion 100 for placement of devices and therapeutic aids. More specifically, the opening 102 shown in FIG. 5 is contiguous with a pocket cut into the material of the cushion 100. The opening and pocket 102 may, for example, be sized to allow the insertion of a hot or cold pack, such that both ankles may be exposed to therapeutic ice or heat. Because the support and cushion 100 is made of a relatively insulative foam material, there is a relatively thin web of material above and below the opening and pocket 102, so as to maximize thermal transfer.

In addition to hot and cold packs, the opening and pocket 102 in the support and cushion 100 may be used to house vibrating elements for massage, although in some embodiments, as shown in FIG. 5, a vibrating element 104 may be housed separately in a specially-contoured pocket. A vibrating element 104 typically includes a motor whose output shaft is arranged to cause an element to vibrate, and a battery pack or power source. In some cases, the vibrating element 104 may be configured to accept household power in the form of standard alternating current, and so a power cord may protrude from the support and cushion 100. If a vibrating element 104 is placed permanently within the support and cushion 100, its controls would be either on the surface of cushion 100 or accessibly within the support and cushion 100. In some embodiments, the vibrating element 104 could be activated using a remote control or a computing device that uses a wireless communication protocol to communicate with the vibrating element.

While the cushions 10, 100 of FIGS. 1-5 are shown without a cover, these cushions 10, 100 may be covered with a fitted fabric cover, or a cover made of another flexible material. The cover may or may not be removable, depending on the embodiment. If the cover is removable, it could include a closure that is secured with, for example, VELCRO® hook-and-loop fasteners or a zipper. In medical or other shared-use embodiments, the cover could be made of a substantially fluid-impregnable material, or one that is otherwise easily cleaned and disinfected. In general use embodiments, the fabric could have any desired decorative pattern or patterns.

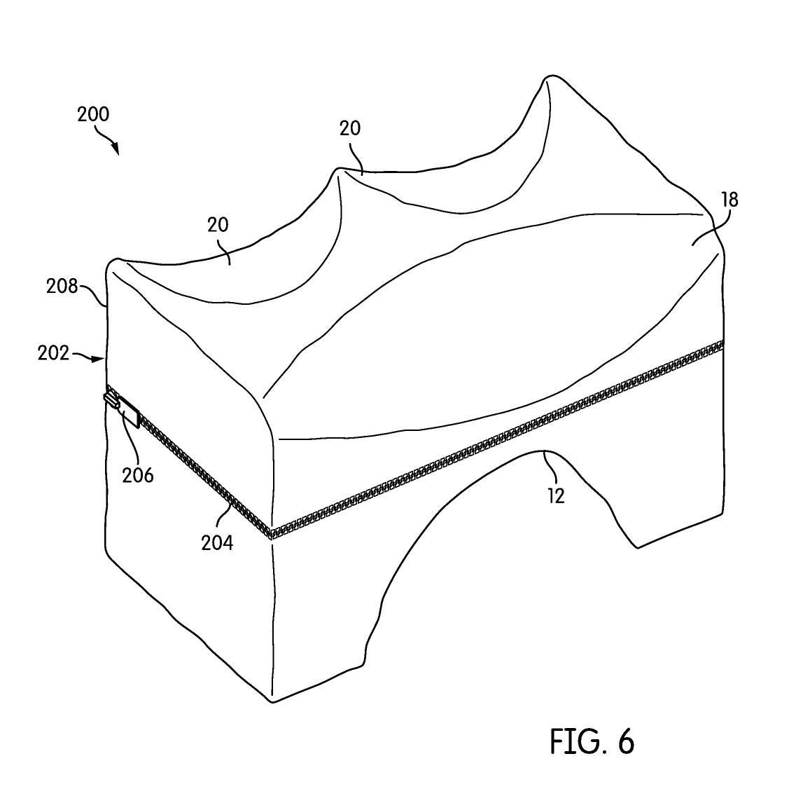

One example of such an arrangement is shown in FIG. 6, which is a perspective view of a support and cushion 200 with a cover 202. The cover 202 is removable and includes a zipper track 204 that extends around three sides of the cushion 200, leaving the fourth side to act as a flexible hinge. To remove the cover, the zipper pull 206 is used to open the cover 202, and the top portion 208 of the cover 202 is swung open.

In some cases, the support and cushion 10, 100, 200 may be defined not by the shape of a foam block or other material that holds its own shape inside a cover, but by the shape of the cover 202 itself. For example, a support and cushion could be comprised of a fabric cover that is sewn to have the features illustrated in FIGS. 1-6 and stuffed with a loose stuffing material, such as polyester batting or rags. In yet other embodiments, the support and cushion could be made of a particularly stiff material, like metal; wood; or contoured, laminate wood. If made of such a stiff material, padded layers would generally be installed over the structural material, and beneath any outer covering, to provide appropriate levels of cushioning.

While the invention has been described with respect to certain embodiments, the embodiments are intended to be exemplary, rather than limiting. Modifications and changes may be made within the scope of the invention, which is defined by the appended claims.

What is claimed is:

1. A support and cushion, comprising:
a channel that extends from a first side face through the body of material to an opposite side face of the body of material, the channel being open at both ends and along a bottom face of the body of material and being of sufficient size to admit at least a lower portion of a leg,
a first elongate concave portion along a first edge of a top portion or element located above the channel, and
a pair of ankle support portions located proximate to a second edge of the top portion opposite the first edge, the ankle support portions being generally concave.

2. The support and cushion of claim 1, wherein the support and cushion comprises a body of a deformable cushioning material.

3. The support and cushion of claim 2, wherein the support and cushion comprises a foam.

4. The support and cushion of claim 3, wherein the foam comprises a urethane foam.

5. The support and cushion of claim 1, wherein the support and cushion is a generally rectilinear block of material.

6. The support and cushion of claim 1, wherein the support and cushion comprises a flexible cover shaped and stuffed to define the channel, the first elongate concave portion, and the pair of ankle support portions.

7. The support and cushion of claim 1, wherein the first side face or the opposite side face includes an opening that is contiguous with a pocket within the body of material.

8. The support and cushion of claim 7, wherein the opening and pocket are sized and adapted to receive a hot or cold pack.

9. A support and cushion, comprising:
a foam cushioning structure defining therein
an channel that extends from a first side face through the foam cushioning structure to an opposite side face of the foam cushioning structure, the channel being open at both ends and along a bottom face of the foam cushioning structure so as to form an open arch in the foam cushioning structure and being of sufficient size to admit at least a lower portion of a leg,
a first elongate concave portion along a first edge of a top portion or element located above the channel, and
a pair of ankle support portions located proximate to a second edge of the top portion opposite the first edge, the ankle support portions being generally concave;
wherein the first elongate concave portion and the pair of ankle support portions are aligned between the first and second side faces and extend in generally the same direction as the channel.

10. The support and cushion of claim 9, wherein the foam cushioning structure comprises a urethane foam.

11. The support and cushion of claim 9, wherein the foam cushioning structure is a generally rectilinear block of material.
